(19) United States
(12) Patent Application Publication
(10) Pub. No.: US 2003/0190712 A1
(43) Pub. Date: Oct. 9, 2003

(54) Fermentation process for the preparation of L-threonine

(75) Inventors: Thomas Hermann, Bielefeld (DE); Mechthild Rieping, Bielefeld (DE)

Correspondence Address: PILLSBURY WINTHROP, LLP, P.O. BOX 10500, MCLEAN, VA, 22102, US

(73) Assignee: Degussa AG

(21) Appl. No.: 10/347,492

(22) Filed: Jan. 21, 2003

Related U.S. Application Data
(63) Continuation-in-part of application No. 09/935,758, filed on Aug. 24, 2001, now Pat. No. 6,562,601.
(60) Provisional application No. 60/236,039, filed on Sep. 28, 2000.

(30) Foreign Application Priority Data
Aug. 31, 2000 (DE) DE 100 42 745.6
Jan. 27, 2001 (DE) DE 101 03 778.3

(51) Int. Cl. C12P 13/04; C12N 15/74; C12N 1/21; C12N 9/06

(52) U.S. Cl. 435/106; 435/252.33; 435/471; 435/191

(57) ABSTRACT

The invention relates to a process for the fermentative preparation of L-threonine in which an L-threonine-producing microorganism of the Enterobacteriaceae family is cultured by the feed process, and a portion of the fermentation broth is then separated off in order to be utilized for inoculation of further media.

### FIELD OF THE INVENTION

The invention provides a new process for the fermentative preparation of L-threonine with Enterobacteriaceae.

### PRIOR ART

L-Threonine is used in animal nutrition, in human medicine and in the pharmaceuticals industry.

It is known that L-threonine can be prepared by fermentation of strains of the Enterobacteriaceae family, in particular Escherichia coli. Because of the great importance of this amino acid, work is constantly being undertaken to improve the preparation processes. Improvements to the process can relate to fermentation measures, such as e.g. stirring and supply of oxygen, or the composition of the nutrient media, such as e.g. the sugar concentration during the fermentation, or the working up to the product form, by e.g. ion exchange chromatography, or the intrinsic output properties, i.e. those of genetic origin, of the microorganism itself.

It is known from the prior art, such as is described, for example, in U.S. Pat. No. 5,538,873 and in EP-B-0593792 or by Okamoto et al. (Bioscience, Biotechnology, and Biochemistry 61 (11), 1877-1882, 1997), that threonine is prepared by fermentation in the batch process (batch) or feed process (fed batch).

### OBJECT OF THE INVENTION

The inventors had the object of providing new measures for improved fermentative preparation of L-threonine.

### SUMMARY OF THE INVENTION

The invention provides a fermentation process, which is characterized in that

a) an L-threonine-producing microorganism of the Enterobacteriaceae family is cultured by the feed process (fed batch) in a known manner, subsequently

b) a portion of the fermentation broth is separated off, 1 to 90 vol. %, in particular 1 to 50 vol. %, preferably 1 to 25 vol. % and particularly preferably 5 to 50 vol. % of the total volume of the fermentation broth remaining in the fermentation tank, subsequently

c) the remaining fermentation broth is topped up with growth medium and, preferably after a growth phase, a further fermentation is carried out by the feed process (fed batch) mentioned,

d) steps b) and c) are optionally carried out several times, and

e) the L-threonine is isolated from the fermentation broths collected.

### DETAILED DESCRIPTION OF THE INVENTION

The microorganisms with which the process according to the invention can be carried out can prepare L-threonine from glucose, sucrose, lactose, fructose, maltose, molasses, starch, or from glycerol and ethanol, the preparation from glucose, sucrose or molasses being preferred. They are representatives of Enterobacteriaceae, in particular of the genera Escherichia, Serratia and Providencia. Of the genus Escherichia the species Escherichia coli and of the genus Serratia the species Serratia marcescens are to be mentioned in particular.

Suitable L-threonine-producing strains of the genus Escherichia, in particular of the species Escherichia coli, are, for example

Escherichia coli TF427

Escherichia coli H-4225

Escherichia coli H-4226

Escherichia coli H-4257

Escherichia coli H-4258

Escherichia coli H-4435

Escherichia coli H-4436

Escherichia coli H-4578

Escherichia coli H-4581

Escherichia coli H-7256

Escherichia coli H-7263

Escherichia coli H-7293

Escherichia coli H-7294

Escherichia coli H-7700

Escherichia coli H-7729

Escherichia coli H-8309

Escherichia coli H-8311

Escherichia coli H-9244

Escherichia coli KY10935

Escherichia coli EL1003

Escherichia coli VNIIgenetika MG-442

Escherichia coli VNIIgenetika VL334/pYN7

Escherichia coli VNIIgenetika M1

Escherichia coli VNIIgenetika 472T23

Escherichia coli VNIIgenetika TDH-6

Escherichia coli BKIIM B-3996

Escherichia coli BKIIM B-5318

Escherichia coli B-3996-C43

Escherichia coli B-3996-C80

Escherichia coli B-3996/pTWV-pps

Escherichia coli B-3996(pMW::THY)

Escherichia coli B-3996/pBP5

Escherichia coli Ferm BP-3756

Escherichia coli Ferm BP-4072

Escherichia coli Ferm BP-1411

Escherichia coli kat 13

Escherichia coli KCCM-10132

Escherichia coli KCCM-10133.

Suitable L-threonine-producing strains of the genus Serratia, in particular of the species Serratia marcescens, are, for example

Serratia marcescens HNr21

Serratia marcescens TLr156

Serratia marcescens T2000

Strains from the Enterobacteriaceae family which produce L-threonine preferably have, inter alia, one or more genetic or phenotypic features chosen from the group consisting of: resistance to α-amino-β-hydroxyvaleric acid, resistance to thialysine, resistance to ethionine, resistance to α-methylserine, resistance to diaminosuccinic acid, resistance to α-aminobutyric acid, resistance to borrelidin, resistance to rifampicin, resistance to valine analogues, such as, for example, valine hydroxamate, resistance to purine analogues, such as, for example, 6-dimethylaminopurine, a need for L-methionine, optionally a partial and compensatable need for L-isoleucine, a need for meso-diaminopimelic acid, auxotrophy in respect of threonine-containing dipeptides, resistance to L-threonine, resistance to L-homoserine, resistance to L-lysine, resistance to L-methionine, resistance to L-glutamic acid, resistance to L-aspartate, resistance to L-leucine, resistance to L-phenylalanine, resistance to L-serine, resistance to L-cysteine, resistance to L-valine, sensitivity to fluoropyruvate, defective threonine dehydrogenase, optionally an ability for sucrose utilization, enhancement of the threonine operon, enhancement of homoserine dehydrogenase I-aspartate kinase I, preferably of the feed back resistant form, enhancement of homoserine kinase, enhancement of threonine synthase, enhancement of aspartate kinase, optionally of the feed back resistant form, enhancement of aspartate semialdehyde dehydrogenase, enhancement of phosphoenol pyruvate carboxylase, optionally of the feed back resistant form, enhancement of phosphoenol pyruvate synthase, enhancement of transhydrogenase, enhancement of the RhtB gene product, enhancement of the RhtC gene product, enhancement of the YfiK gene product, enhancement of a pyruvate carboxylase, and attenuation of acetic acid formation.

Thus, for example, the strain 472T23 (U.S. Pat. No. 5,631,157) has, inter alia, an enhanced, “feed back” resistant aspartate kinase I-homoserine dehydrogenase I, an attenuated threonine deaminase, a partial need for L-isoleucine, a resistance to at least 5 g/l L-threonine, a resistance to homoserine and the ability to utilize sucrose as a source of carbon.

Thus, for example, the strain B-3996 (U.S. Pat. No. 5,175,107) has, inter alia, an enhanced, “feed back” resistant aspartate kinase I-homoserine dehydrogenase I, an attenuated threonine deaminase, a partial need for L-isoleucine, an attenuated threonine dehydrogenase, a resistance to at least 5 g/l L-threonine and the ability to utilize sucrose as a source of carbon.

Thus, for example, the strain kat-13 (U.S. Pat. No. 5,939,307) has, inter alia, an enhanced, “feed back” resistant aspartate kinase I-homoserine dehydrogenase I, an attenuated threonine dehydrogenase, resistance to borrelidin and the ability to utilize sucrose as a source of carbon.

Thus, for example, the strain KCCM-10132 (WO 00/09660) has, inter alia, a resistance to α-methylserine, a resistance to diaminosuccinic acid, sensitivity to fluoropyruvate, a resistance to L-glutamic acid and a resistance to at least 7% L-threonine. The strain is also in need of the amino acids L-methionine and L-isoleucine. It was found out that strain KCCM-10132 has a feed back” resistant aspartate kinase I-homoserine dehydrogenase I.

Thus, for example, the strain H-4581 (U.S. Pat. No. 4,996,147) has, inter alia, a resistance to rifampicine, a resistance to L-lysine, a resistance to L-methionine, a resistance to aspartic acid, a resistance to homoserine and a need for m-diaminopimelic acid. It was found out that strain H-4581 has a “feed back” resistant aspartate kinase I-homoserine dehydrogenase I.

The term “feed back” resistant aspartate kinase I-homoserine dehydrogenase I denotes an enzyme mutant variant which is less susceptible to inhibition by L-threonine as compared to the wild type enzyme. Microorganism containing “feed back” resistant aspartate kinase I-homoserine dehydrogenase I are typically resistant to the threonine analogue α-amino-β-hydroxyvaleric acid (AHV). Instructions concerning α-amino-β-hydroxyvaleric acid, growth inhibition exerted by AHV, the enzyme aspartate kinase I-homoserine dehydrogenase I and threonine insensitive enzyme variants are described in the art for example by Shio and Nakamori (Agricultural and Biological Chemistry 33(8), 1152-1160 (1969)), Lee et al. (Korean Journal of Applied Microbiology and Biotechnology 19(6), 583-587 (1991)), Saint-Girons and Margerita (Molecular and General Genetics 162, 101-107 (1978)), U.S. Pat. No. 4,278,765, Fazel et al. (Journal of Biological Chemistry 258(22), 13570-13574 (1983)), Omori et al. (Journal of Bacteriology 175(3), 785-794 (1993)), Omori et al. (Journal of Bacteriology 175(4), 959-965 (1993)) or Neidhard et al. (Escherichia coli and Salmonella. Cellular and Molecular Biology (ASM Press, Washington D.C., 1996)).

The term “enhancement” in this connection describes the increase in the intracellular activity or concentration of one or more enzymes or proteins in a microorganism which are coded by the corresponding DNA, for example by increasing the number of copies of the gene or allele or of the genes or alleles, using a plasmid, using a potent promoter or using a gene or allele which codes for a corresponding enzyme having a high activity, and optionally combining these measures.

By enhancement measures, in particular over-expression, the activity or concentration of the corresponding protein is in general increased by at least 10%, 25%, 50%, 75%, 100%, 150%, 200%, 300%, 400% or 500%, up to a maximum of 1000% or 2000%, based on the starting microorganism.

The term “attenuation” in this connection describes the reduction or elimination of the intracellular activity or concentration of one or more enzymes or proteins in a microorganism which are coded by the corresponding DNA, for example by using a weak promoter or using a gene or allele which codes for a corresponding enzyme with a low activity or inactivates the corresponding gene or enzyme or protein, and optionally combining these measures.

By attenuation measures, the activity or concentration of the corresponding protein is in general reduced to 0 to 50%, 0 to 25%, 0 to 10% or 0 to 5% of the activity or concentration of the wild-type protein.

According to the invention, the system output of a fermentation unit producing L-threonine is increased by a procedure in which after a first fermentation step a portion of the fermentation broth obtained in this way remains in the production fermenter and serves as the inoculum for one or more further fermentation steps (batches).

According to the invention, 1 to 90 vol. %, preferably 1 to 50 vol. %, preferentially 1 to 25 vol. %, 1 to 20 vol. %, 1 to 15 vol. % or 1 to 10 vol. %, and particularly preferably 5 to 20 vol. %, 5 to 15 vol. % or 1 to 10 vol. % of the total volume of the fermentation broth remains in the fermentation tank.

The broth remaining in the fermentation tank is preferably topped up with a growth medium. After optionally >0 to not more than 10 hours, preferably after 1 to 10 hours, preferentially 2 to 10 hours and particularly preferably 3 to 7 hours a production medium is fed in. Alternatively, the components of this medium can also be fed in separately. After 20 to 72 hours, preferably 20 to 48 hours, the batch is ended and a portion of the fermentation broth, as described above, is separated off. A new fermentation stage is then optionally started with the remainder. The process can be repeated at least once, preferably approx. 2 to 6 times, depending on the stability of the strain used. Repetitions of approx. 2 to 8 times or 2 to 10 times or 2 to 4 times are also possible.

The L-threonine producing microorganisms which can preferrably be used for the process according to the invention comprise Escherichia coli strains having at least a “feed back” resistant aspartate kinase I-homoserine dehydrogenase I.

In addition the strains may comprise one or more of the features selected from the group consisting of an attenuated threonine deaminase, a partial need for L-isoleucine, a resistance to at least 5 g/l L-threonine, a resistance to homoserine, the ability to utilize sucrose as a source of carbon, an attenuated threonine dehydrogenase, a resistance to borrelidin, a resistance to α-methylserine, a resistance to diaminosuccinic acid, sensitivity to fluoropyruvate, a resistance to L-glutamic acid, a resistance to at least 7% L-threonine, a need for L-methionine, a need for L-isoleucine a resistance to rifampicine, a resistance to L-lysine, a resistance to L-methionine, a resistance to aspartic acid and a need for m-diaminopimelic acid.

Appropriately stable strains which do not lose their production properties in the course of the process are particularly suitable for the process described.

Strains which can be used for the process described may contain a plasmid comprising at least one of the genes selected from the group consisting of a gene encoding homoserine dehydrogenase I-aspartate kinase I, preferably of the “feed back” resistant form, a gene encoding homoserine kinase and a gene encoding threonine synthase. These plasmids may for example originate from pBR322 (Bolivar et al., Gene 2, 95-113 (1977)), pAYC32 (Chistoserdov and Tsygankov, Plasmid 16, 161-167 (1986)), pSC101 (Bernardi and Bernardi, Nucleic Acids Research 12(24), 9415-9426 (1984)) or other plasmids described in the art.

Such plasmids are optionally stabilized by one or more of the genetic loci selected from the group consisting of the parB locus of plasmid R1 described by Rasmussen et al. (Molecular and General Genetics 209 (1), 122-128 (1987)), Gerdes et al. (Molecular Microbiology 4 (11), 1807-1818 (1990)) and Thistedt and Gerdes (Journal of Molecular Biology 223 (1), 41-54 (1992)), the flm locus of the F plasmid described by Loh et al. (Gene 66 (2), 259-268 (1988)), the par locus of plasmid pSC101 described by Miller et al. (Gene 24 (2-3), 309-315 (1983), the cer locus of plasmid ColE1 described by Leung et al. (DNA 4 (5), 351-355 (1985), the par locus of plasmid RK2 described by Sobecky et al. (Journal of Bacteriology 178 (7), 2086-2093 (1996)) and Roberts and Helinsky (Journal of Bacteriology 174 (24), 8119-8132 (1992)), the par locus of plasmid RP4 described by Eberl et al. (Molecular Microbiology 12 (1), 131-141 (1994)) and the parA locus of plasmid R1 described by Gerdes and Molin (Journal of Molecular Biology 190 (3), 269-279 (1986)), Dam and Gerdes (Journal of Molecular Biology 236 (5), 1289-1298 (1994)) and Jensen et al (Proceedings of the National Academy of Sciences USA 95 (15), 8550-8555 (1998).

The growth medium typically comprises sugars, such as e.g. glucose, starch hydrolysate, sucrose or molasses, as the source of carbon. Organic nitrogen-containing compounds, such as peptones, yeast extract, meat extract, malt extract, corn steep liquor, soya bean flour and urea, or inorganic compounds, such as ammonium sulfate, ammonium chloride, ammonium phosphate, ammonium carbonate and ammonium nitrate, can be used as the source of nitrogen. The sources of nitrogen can be used individually or as a mixture. Phosphoric acid, potassium dihydrogen phosphate or dipotassium hydrogen phosphate or the corresponding sodium-containing salts can be used as the source of phosphorus. The culture medium must furthermore comprise salts of metals, such as e.g. magnesium sulfate, manganese sulfate or iron sulfate, which are necessary for growth. Finally, essential growth substances, such as amino acids (e.g. homoserine) and vitamins (e.g. thiamine), are employed in addition to the above-mentioned substances. Antifoams, such as e.g. fatty acid polyglycol esters, can be employed to control the development of foam.

In general, the production medium comprises only one sugar, such as e.g. sucrose or glucose, and optionally an inorganic source of nitrogen, such as e.g. ammonium sulfate. Alternatively, these and other components can also be fed in separately.

During the growth or production phase, the temperature is established in a range from 29° C. to 42° C., preferably 33° C. to 40° C. Temperatures in a range from 27° C. to 39° C. are also possible. The fermentation can be carried out under normal pressure or optionally under increased pressure, preferably under an increased pressure of 0 to 1.5 bar. The oxygen partial pressure is regulated at 5 to 50%, preferably approx. 20% atmospheric saturation. Regulation of the pH to a pH of approx. 6 to 8, preferably 6.5 to 7.5, can be effected with 25% aqueous ammonia.

The process according to the invention is distinguished with respect to conventional processes above all by an increased space/time yield or productivity.

The present invention is explained in more detail in the following with the aid of embodiment examples.

The isolation of plasmid DNA from Escherichia coli and all techniques of restriction, Klenow and alkaline phosphatase treatment were carried out by the method of Sambrook et al. (Molecular Cloning. A laboratory manual (1989) Cold Spring Harbor Laboratory Press). Unless described otherwise, the transformation of Escherichia coli was carried out by the method of Chung et al. (Proceedings of the National Academy of Sciences of the United States of America USA (1989) 86: 2172-2175).

### EXAMPLE 1

Preparation of the Escherichia coli K-12 Strain DM1265

A plasmid-free variant of the E. coli strain 472T23 was obtained from the American Type Culture Collection (Manasas, Va., USA) as ATCC98082. The strain ATCC98082 is described in the patent specification U.S. Pat. No. 5,631,157. The E. coli strain VL334/pYN7 was obtained from the Russian National Collection of Industrial Microorganisms (VKPM, Moscow, Russia) as CMIM B-1684. The strain CMIM B-1684 is described in the patent specification U.S. Pat. No. 4,278,765.

The plasmid pYN7 was isolated from the strain VL334/pYN7. A DNA fragment 6.25 kbp long which carries the thrABC operon was isolated from plasmid pYN7 by preparative agarose gel electrophoresis with the aid of the restriction enzymes HindIII and BamHI.

The plasmid pBR322 (Bolivar et al., Gene 2, 95-113 (1977)) was obtained from Pharmacia Biotech (Uppsala, Sweden) and treated with the restriction enzymes HindIII and BamHI. The DNA fragment 4.3 kbp long was isolated by preparative agarose gel electrophoresis. The two DNA fragments were mixed, treated with T4 DNA ligase, and the strain DH5α was transformed with the ligation mixture. After selection on ampicillin-containing (50 μg/mL) LB agar, transformants which contained a plasmid which corresponded in its structure to the plasmid pYN7 were obtained.

The plasmid was isolated from a transformant, cleaved partly with the enzyme EcoRI and completely with the enzyme HindIII and ligated with the parB gene region isolated. For this, the plasmid pKG1022 (Gerdes, Biotechnology (1988) 6:1402-1405) was cleaved with the enzymes EcoRI and HindIII, the cleavage batch was separated in 1% agarose gel and the parB fragment 629 bp in size was isolated with the aid of the QIAquick Gel Extraction Kit (QIAGEN, Hilden, Germany). The ligation mixture was employed for transformation of strain ATCC98082. Selection of plasmid-carrying cells was carried out on LB agar (Lennox, Virology 1:190 (1955)), to which 50 μg/ml ampicillin had been added. Successful cloning of the parB gene region could be detected after isolation of the plasmid DNA, control cleavage with EcoRI and HindIII and analysis of the cleavage batch by agarose gel electrophoresis. The plasmid was designated pYN7parB.

A transformant of the type ATCC98082/pYN7parB has been designated DM1265 and deposited in the form of a pure culture on Apr. 30, 1999 at the Deutsche Sammlung fur Mikroorganismen und Zellkulturen (German Collection of Microorganisms and Cell Cultures=DSM, Braunschweig, Germany) as DSM12790 in accordance with the Budapest Treaty.

The strain DM1265 has, inter alia, an enhanced, “feed back” resistant aspartate kinase I-homoserine dehydrogenase I, an attenuated threonine deaminase, a resistance to at least 5 g/l L-threonine and the ability to utilize sucrose as a source of carbon.

This strain is distinguished by a high stability, in particular segregation stability.

### COMPARATIVE EXAMPLE A

Preparation of L-Threonine with the Aid of the Escherichia coli K-12 Strain DM1265 by Conventional Fermentation

An individual colony of the strain DM1265 was transinoculated on to minimal medium with the following composition: 3.5 g/l Na₂HPO₄*2H₂O, 1.5 g/l KH₂PO₄, 1 g/l NH₄Cl, 0.1 g/l MgSO₄*7H₂O, 2 g/l sucrose, 20 g/l agar, 50 mg/l ampicillin. The culture was incubated at 37° C. for approx. 5 days. 10 ml preculture medium with the following composition: 2 g/l yeast extract, 10 g/l (NH₄)₂SO₄, 1 g/l KH₂PO₄, 0.5 g/l MgSO₄*7H₂O, 15 g/l CaCO₃, 20 g/l sucrose, 50 mg/l ampicillin were inoculated with an inoculating loop and incubated for 16 (h) at 37° C. and 180 rpm on an ESR incubator from Kuhner AG (Birsfelden, Switzerland).

A volume of 1 ml of this first preculture was inoculated into 1402 g of the nutrient medium A1-144. The culturing fermentation was carried out in 2 l stirred reactor fermenters from B. Braun (BBI, Germany, Melsungen, Biostat MD model). The nutrient medium A1-144 contained the constituents listed in Table 1. This second preculture was cultured for 22.5 h at a temperature of 37° C., a volume-specific gassing of 0.71 vvm (volume per volume per minute), an oxygen partial pressure of 10% of the atmospheric saturation and a pH of pH 7.0 until an optical density (OD) (660 nm) of 16.3 was reached.

For inoculation of 1233 g of the growth medium M1-463, which was contained in 2 l stirred reactor fermenters from B. Braun (BBI, Germany, Melsungen, Biostat MD model), 157.6 g of the second preculture in nutrient medium A1-144 were added. The growth medium M1-463 contained the constituents listed in Table 2. The culture was cultured at a temperature of 37° C., an aeration of 1 l/min, a minimum stirring of 800 rpm and a pH of 7.0 and an oxygen partial pressure of 20% of the atmospheric saturation until a residual sugar concentration of approx. 3 g/l was reached. The broth obtained in this way was subsequently cultured for a further 30 h at a temperature of 37° C., an oxygen partial pressure of 20% of the atmospheric saturation and a pH of pH 7.0 until an OD (660 nm) of 33.4 was reached. During this time, 450 g of a production medium comprising a sucrose solution with a concentration of 650 g/l was fed in continuously.

The optical density (OD) was then determined with a digital photometer of the LP1W type from Dr. Bruno Lange GmbH (Berlin, Germany) at a measurement wavelength of 660 nm and the concentration of L-threonine formed was determined by ion exchange chromatography and post-column reaction with ninhydrin detection with an amino acid analyzer from Eppendorf-BioTronik (Hamburg, Germany),

After 39.5 h, an L-threonine concentration of 69.6 g/l was found in the final fermentation sample. The space/time yield in this experiment was thus 1.76 g/l·h.

TABLE 1
Composition of nutrient medium A1-144

| | |
|---|---|
| Component | Concentration (per kg) |

| | | |
|---|---|---|
| Sucrose | 30 | g |
| Yeast extract | 2 | g |
| (NH₄)₂SO₄ | 5 | g |
| K₂HPO₄ | 2 | g |
| NaCl | 0.6 | g |
| MgSO₄ · 7H₂O | 0.4 | g |
| FeSO₄ · 7H₂O | 20 | mg |
| MnSO₄ · H₂O | 20 | mg |
| Ampicillin | 50 | mg |
| Structol | 0.3 | g |

TABLE 2
Composition of nutrient medium M1-463

| | |
|---|---|
| Component | Concentration (per kg) |

| | | |
|---|---|---|
| Sucrose | 27.7 | g |
| Yeast extract | 1.87 | g |
| NaCl | 0.62 | g |
| (NH₄)₂SO₄ | 4.7 | g |
| K₂HPO₄ | 1.9 | g |
| MgSO₄ · 7H₂O | 0.38 | g |
| MnSO₄ · H₂O | 18 | mg |
| FeSO₄ · 7H₂O | 18 | mg |
| Ampicillin | 50 | mg |
| Structol | 0.1 | g |

### EXAMPLE 2

Preparation of L-Threonine with the Aid of the Strain DM1265 with 2 Subsequent Feed Processes and 10% Inoculum in Each Case

An individual colony of the strain DM1265 was transinoculated on to minimal medium with the following composition: 3.5 g/l Na₂HPO₄*2H₂O, 1.5 g/l KH₂PO₄, 1 g/l NH₄Cl, 0.1 g/l MgSO₄*7H₂O, 2 g/l glucose, 20 g/l agar, 50 mg/l ampicillin. The culture was incubated at 37° C. for approx. 5 days. 10 ml preculture medium with the following composition: 2 g/l yeast extract, 10 g/l (NH₄)₂SO₄, 1 g/l KH₂PO₄, 0.5 g/l MgSO₄*7H₂O, 15 g/l CaCO₃, 20 g/l glucose, 50 mg/l ampicillin were inoculated with an inoculating loop and incubated for 16 h at 37° C. and 180 rpm on an ESR incubator from Kühner AG (Birsfelden, Switzerland).

A volume of 1 ml of this first preculture was inoculated into 1402 g of the nutrient medium A1-144. The culturing fermentation was carried out in 2 l stirred reactor fermenters from B. Braun (BBI, Germany, Melsungen, Biostat MD model). The nutrient medium A1-144 contained the constituents listed in Table 1. This second preculture was cultured for 22.5 h at a temperature of 37° C., a volume-specific gassing of 0.71 vvm, an oxygen partial pressure of 10% of the atmospheric saturation and a pH of pH 7.0 until an OD (660 nm) of 16.3 was reached.

For inoculation of 1233 g of the growth medium M1-463, which was contained in 2 l stirred reactor fermenters from B. Braun (BBI, Germany, Melsungen, Biostat MD model), 157.6 g of the second preculture in nutrient medium A1-144 were added. The growth medium M1-463 contained the constituents listed in Table 2. The culture was cultured as described in Comparative Example A at a temperature of 37° C., an aeration of 1 l/min, a minimum stirring of 800 rpm and a pH of 7.0 and an oxygen partial pressure of 20% of the atmospheric saturation until a residual sugar concentration of approx. 3 g/l was reached after 9.5 h. The fermentation broth obtained in this way was then cultured for a further 30 h at a temperature of 37° C., an oxygen partial pressure of 20% of the atmospheric saturation and a pH of pH 7.0 until an OD (660 nm) of 33.4 was reached. During this time, 450 g of a production medium comprising a sucrose solution with a concentration of 650 g/l was fed in continuously. After the feed solution had been consumed and the residual sugar in the fermentation broth of this first run had been consumed, 90% of the fermentation broth (1656 g) of the fermenter contents was removed by pumping off.

The remaining 10% of the volume (184 g) was topped up with 1200 g of the growth medium M1-474 and the fermentation was started again. The growth medium M1-474 contained the constituents listed in Table 3. The culture of this second run was cultured as described in Comparative Example A at a temperature of 37° C., an aeration of 1 l/min, a minimum stirring of 800 rpm and a pH of 7.0 and an oxygen partial pressure of 20% of the atmospheric saturation until a residual sugar concentration of approx. 3 g/l was reached after 5 h. The broth was then cultured for a further 31.25 h at a temperature of 37° C., an oxygen partial pressure of 20% of the atmospheric saturation and a pH of pH 7.0 until an OD (660 nm) of 35.7 was reached. During this time, 450 g of a production medium comprising a sucrose solution with a concentration of 650 g/l was fed in continuously. After the feed solution had been consumed and the residual sugar in the fermentation broth had been consumed, 90% of the fermentation broth (1656 g) was removed from the fermenter by pumping off.

The remaining 10% of the total amount (184 g) was topped up with 1200 g of the growth medium M1-474 and the fermentation was started again. The growth medium M1-474 contained the constituents listed in Table 3. The culture of this third run was cultured as described in Comparative Example A at a temperature of 37° C., an aeration of 1 l/min, a minimum stirring of 800 rpm and a pH of 7.0 and an oxygen partial pressure of 20% of the atmospheric saturation until a residual sugar concentration of approx. 3 g/l was reached after 5.25 h. The culture was then cultured for a further 30.5 h at a temperature of 37° C., an oxygen partial pressure of 20% of the atmospheric saturation and a pH of pH 7.0 until an OD (660 nm) of 32.5 was reached. During this time, 450 g of a production medium comprising a sucrose solution with a concentration of 650 g/l was fed in.

At the end of each fermentation the OD and the concentration of L-threonine formed were determined as in Comparative Example A. The results of the particular runs are shown in Table 4.

The term “space/time” yield here describes the volumetric productivity, i.e. the quotient of the concentration of L-threonine at the end of the fermentation and the fermentation time.

TABLE 3
Composition of nutrient medium M1-474

| | |
|---|---|
| Component | Concentration (per kg) |

| | | |
|---|---|---|
| Sucrose | 27.7 | g |
| Yeast extract | 1.68 | g |
| NaCl | 0.62 | g |
| (NH₄)₂SO₄ | 4.7 | g |
| K₂HPO₄ | 1.9 | g |
| MgSO₄ · 7H₂O | 0.38 | g |
| MnSO₄ · H₂O | 18 | mg |
| FeSO₄ · 7H₂O | 18 | mg |
| Ampicillin | 50 | mg |
| Structol | 0.1 | g |

TABLE 4
Results from Example 2

| | | | OD | Space/time |
|---|---|---|---|---|
| | | L-Threonine | (660 | yield |
| Run | Time [h] | [g/l] | nm) | [g/l · h] |
| 1 | 39.5 | 68.4 | 33.4 | 1.73 |
| 2 | 36.25 | 69.8 | 35.7 | 1.93 |
| 3 | 35.75 | 69.2 | 32.5 | 1.94 |

### EXAMPLE 3

Preparation of L-threonine with the aid of the strain DM1265 with 4 subsequent feed processes and 25% inoculation in each case

An individual colony of the strain DM1265 was transinoculated on to minimal medium with the following composition: 3.5 g/l Na₂HPO₄*2H₂O, 1.5 g/l KH₂PO₄, 1 g/l NH₄Cl, 0.1 g/l MgSO₄*7H₂O, 2 g/l glucose, 20 g/l agar, 50 mg/l ampicillin. The culture was incubated at 37° C. for approx. 5 days. 10 ml preculture medium with the following composition: 2 g/l yeast extract, 10 μl (NH₄)₂SO₄, 1 g/l KH₂PO₄, 0.5 g/l MgSO₄*7H₂O, 15 μl CaCO₃, 20 g/l glucose, 50 mg/l ampicillin were inoculated with an inoculating loop and incubated for 16 h at 37° C. and 180 rpm on an ESR incubator from Kuhner AG (Birsfelden, Switzerland).

A volume of 1 ml of this first preculture was inoculated into 1402 g of the nutrient medium A1-144. The culturing fermentation was carried out in 2 l stirred reactor fermenters from B. Braun (BBI, Germany, Melsungen, Biostat MD model). The nutrient medium A1-144 contained the constituents listed in Table 1. This second preculture was cultured for 22.5 h at a temperature of 37° C., a volume-specific gassing of 0.71 vvm, an oxygen partial pressure of 10% of the atmospheric saturation and a pH of pH 7.0 until an OD (660 nm) of 16.3 was reached.

For inoculation of 1233 g of the growth medium M1-463, which was contained in 2 l stirred reactor fermenters from B. Braun (BBI, Germany, Melsungen, Biostat MD model), 157.6 g of the second preculture in nutrient medium A1-144 were added. The growth medium M1-463 contained the constituents listed in Table 2. The culture was cultured as described in Comparative Example A at a temperature of 37° C., an aeration of 1 l/min, a minimum stirring of 800 rpm and a pH of 7.0 and an oxygen partial pressure of 20% of the atmospheric saturation until a residual sugar concentration of approx. 3 g/l was reached after 9.5 h. The fermentation broth obtained in this way was then cultured for a further 32 h at a temperature of 37° C., an oxygen partial pressure of 20% of the atmospheric saturation and a pH of pH 7.0 until an OD (660 nm) of 35.8 was reached. During this time, 450 g of a production medium comprising a sucrose solution with a concentration of 650 g/l was fed in continuously. After the feed solution had been consumed and the residual sugar in the fermentation broth of this first run had been consumed, 75% of the fermentation broth of the fermenter contents was removed by pumping off. The first fermentation (first run) was ended after 41.5 h and reached a titre of 67.1 g/l threonine.

The remaining 25% of the total amount (453 g) was topped up with 700 g of the growth medium M1-527 and the fermentation was started again. The growth medium M1-527 contained the constituents listed in Table 5. The culture of this second run was cultured as described in Comparative Example A at a temperature of 37° C., an aeration of 1 l/min, a minimum stirring of 800 rpm and a pH of 7.0 and an oxygen partial pressure of 20% of the atmospheric saturation until a residual sugar concentration of approx. 3 g/l was reached. The culture was then cultured for a further 30 h at a temperature of 37° C., an oxygen partial pressure of 20% of the atmospheric saturation and a pH of pH 7.0 until an OD (660 nm) of 36.2 was reached. During this time, 450 g of a production medium comprising a sucrose solution with a concentration of 650 g/l was fed in as in the first run. The draining off of the fermentation broth to 25% and the topping up of the fermenter with M1-527 was repeated a total of four times.

At the end of each fermentation the OD and the concentration of L-threonine formed were determined as in Comparative Example A.

Table 6 shows the results of the particular runs. The term “Total L-threonine formed” relates to the L-threonine effectively formed or produced during the fermentation run. To calculate the total L-threonine formed, the amount of L-threonine introduced by the inoculum is subtracted from the amount of L-threonine present in the fermentation tank at the end of the run. The term “Productivity” designates the quotient of the total L-threonine formed per fermentation run and the fermentation time per run.

TABLE 5
Composition of growth medium M1-527

| | |
|---|---|
| Component | Concentration (per kg) |

| | | |
|---|---|---|
| Sucrose | 34.10 | g |
| Yeast extract | 2.31 | g |
| NaCl | 0.76 | g |
| (NH₄)₂SO₄ | 5.78 | g |
| K₂HPO₄ | 2.314 | g |
| MgSO₄ · 7H₂O | 0.464 | g |
| MnSO₄ · H₂O | 22.7 | mg |
| FeSO₄ · 7H₂O | 22.7 | mg |
| Ampicillin | 60 | mg |
| Structol | 120 | g |

TABLE 6
Results from Example 3

| | | | | Total L- | |
|---|---|---|---|---|---|
| | | | OD | Threonine | Product- |
| | | L-Threonine | (660 | formed | ivity |
| Run | Time [h] | [g/l] | nm) | [g] | [g/h] |
| 1 | 41.5 | 67.1 | 35.8 | 120.7 | 2.91 |
| 2 | 35.7 | 74.5 | 36.2 | 100.5 | 2.82 |
| 3 | 34.5 | 80.0 | 31.4 | 108.0 | 3.13 |
| 4 | 35.0 | 76.3 | 33.4 | 103.0 | 2.94 |
| 5 | 41.5 | 73.0 | 31.7 | 98.6 | 2.37 |

### COMPARATIVE EXAMPLE B

Preparation of L-Threonine with the Aid of the Escherichia coli K-12 Strain Kat-13 by Conventional Fermentation

The L-threonine-producing E. coli strain kat-13 is described in U.S. Pat. No. 5,939,307 and deposited at the Agriculture Research Service Patent Culture Collection (Peoria, Ill., USA) as NRRL B-21593.

The strain kat-13 has, inter alia, an enhanced, “feed back” resistant aspartate kinase I-homoserine dehydrogenase I, an attenuated threonine dehydrogenase, resistance to borrelidin and the ability to utilize sucrose as a source of carbon.

An individual colony of the strain kat-13 was transinoculated on to minimal medium with the following composition: 3.5 g/l Na₂HPO₄*2H₂O, 1.5 g/l KH₂PO₄, 1 g/l NH₄Cl, 0.1 g/l MgSO₄*7H₂O, 2 g/l glucose, 20 g/l agar. The culture was incubated at 37° C. for approx. 5 days. 10 ml preculture medium with the following composition: 2 g/l yeast extract, 10 g/l (NH₄)₂SO₄, 1 g/l KH₂PO₄, 0.5 g/l MgSO₄*7H₂O, 15 g/l CaCO₃, 20 g/l glucose, were inoculated with an inoculating loop and incubated for 16 h at 37° C. and 180 rpm on an ESR incubator from Kuhner AG (Birsfelden, Switzerland).

A volume of 0.45 ml of this first preculture was inoculated into 1500 g of the nutrient medium A1-158. The culturing fermentation was carried out in 2 l stirred reactor fermenters from B. Braun (BBI, Germany, Melsungen, Biostat MD model). The nutrient medium A1-158 contained the constituents listed in Table 7. This second preculture was cultured for 19.75 h at a volume-specific gassing of 1.16 vvm, an oxygen partial pressure of 20% of the atmospheric saturation and a pH of pH 6.9 until all the glucose had been consumed. The fermentation was started at a temperature of 39° C., and after a fermentation time of 18 h the temperature was lowered to 37° C.

For inoculation of 725 g of the growth medium M1-530, which was contained in 2 l stirred reactor fermenters from B. Braun (BBI, Germany, Melsungen, Biostat MD model), 110 g of the second preculture in nutrient medium A1-158 were added. The growth medium M1-530 contained the constituents listed in Table 8. The culture was cultured at a temperature of 37° C., an aeration of 1.3 l/min, a minimum stirring of 800 rpm and a pH of 7.0 and an oxygen partial pressure of 20% of the atmospheric saturation until all the glucose initially introduced had been consumed after 8 h. The fermentation broth obtained in this way was then cultured for a further 57 h at a temperature of 37° C., an oxygen partial pressure of 20% of the atmospheric saturation, an aeration of 1.5 l/min and a pH of pH 7.0. During this time, 1000 g of a production medium comprising a glucose.H₂O solution with a concentration of 550 g/l was fed in continuously.

The OD and the concentration of L-threonine formed were then determined as in Comparative Example A.

After 65 h, an L-threonine concentration of 101.3 g/l was found in the final fermentation sample. The space/time yield in this experiment was thus 1.56 g/l·h.

TABLE 7
Composition of nutrient medium A1-158

| | |
|---|---|
| Component | Concentration (per kg) |

| | | |
|---|---|---|
| Glucose · H₂O | 88 | g |
| Corn steep liquor (50%) | 20 | g |
| (NH₄)₂SO₄ | 0.5 | g |
| KH₂PO₄ | 2.5 | g |
| Citric acid | 0.192 | g |
| MgSO₄ · 7H₂O | 2 | g |
| FeSO₄ · 7H₂O | 30 | mg |
| MnSO₄ · H₂O | 21 | mg |
| Kanamycin | 50 | mg |
| Structol | 0.3 | g |

TABLE 8
Composition of growth medium M1-530

| | |
|---|---|
| Component | Concentration (per kg) |

| | | |
|---|---|---|
| Glucose · H₂O | 88 | g |
| Corn steep liquor (50%) | 20 | g |
| Citric acid | 0.192 | g |
| (NH₄)₂SO₄ | 0.5 | g |
| KH₂PO₄ | 2.5 | g |
| MgSO₄ · 7H₂O | 2.0 | g |
| MnSO₄ · H₂O | 21 | mg |
| FeSO₄ · 7H₂O | 30 | mg |
| Kanamycin | 50 | mg |
| Structol | 0.3 | g |

### EXAMPLE 4

Preparation of L-Threonine with the Aid of the Strain Kat-13 with a Subsequent Feed Process and 10% Inoculation

An individual colony of the strain kat-13 was transinoculated on to minimal medium with the following composition: 3.5 g/l Na₂HPO₄*2H₂O, 1.5 g/l KH₂PO₄, 1 g/l NH₄Cl, 0.1 g/l MgSO₄*7H₂O, 2 g/l glucose, 20 g/l agar. The culture was incubated at 37° C. for approx. 5 days. 10 ml preculture medium with the following composition: 2 g/l yeast extract, 10 g/l (NH₄)₂SO₄, 1 g/1 KH₂PO₄, 0.5 g/l MgSO₄*7H₂O, 15 g/l CaCO₃, 20 g/l glucose, were inoculated with an inoculating loop and incubated for 16 h at 37° C. and 180 rpm on an ESR incubator from Kuhner AG (Birsfelden, Switzerland).

A volume of 0.45 ml of this first preculture was inoculated into 1500 g of the nutrient medium A1-158. The culturing fermentation was carried out in 2 l stirred reactor fermenters from B. Braun (BBI, Germany, Melsungen, Biostat MD model). The nutrient medium A1-158 contained the constituents listed in Table 7. This second preculture was cultured for 19.75 h at a volume-specific gassing of 1.16 vvm, an oxygen partial pressure of 20% of the atmospheric saturation and a pH of pH 6.9 until all the glucose had been consumed. The fermentation was started at a temperature of 39° C., and after a fermentation time of 18 h the temperature was lowered to 37° C.

For inoculation of 725 g of the growth medium M1-530, which was contained in 2 l stirred reactor fermenters from B. Braun (BBI, Germany, Melsungen, Biostat MD model), 110 g of the second preculture in nutrient medium A1-158 were added. The growth medium M1-530 contained the constituents listed in Table 8. The culture was cultured at a temperature of 37° C., an aeration of 1.3 l/min, a minimum stirring of 800 rpm and a pH of 7.0 and an oxygen partial pressure of 20% of the atmospheric saturation until all the glucose initially introduced had been consumed after 8 h. The fermentation broth obtained in this way was then cultured for a further 57 h at a temperature of 37° C., an oxygen partial pressure of 20% of the atmospheric saturation, an aeration of 1.5 l/min and a pH of pH 7.0 until an OD (660 nm) of 46.4 was reached. During this time, 1000 g of a production medium comprising a glucose-H₂O solution with a concentration of 550 g/l was fed in continuously. After the feed solution had been consumed and the residual sugar in the fermentation broth of this first run had been consumed, 90% of the fermentation broth (1651 g) of the fermenter contents was removed by pumping off.

The remaining 10% of the volume (184 g) was topped up with 650 g of the growth medium M1-531 and the fermentation was started again. The growth medium M1-531 contained the constituents listed in Table 9. The culture was cultured at a temperature of 37° C., an aeration of 1.5 l/min, a minimum stirring of 800 rpm and a pH of 7.0 and an oxygen partial pressure of 20% of the atmospheric saturation. During this time, 1000 g of a production medium comprising a glucose.H₂O solution with a concentration of 550 g/l was fed in continuously.

At the end of each fermentation the OD and the concentration of L-threonine formed were determined as in Comparative Example A.

The results of the two runs are shown in Table 10. The term “Total L-threonine formed” relates to the L-threonine effectively formed or produced during the fermentation run. To calculate the total L-threonine formed, the amount of L-threonine introduced by the inoculum is subtracted from the amount of L-threonine present in the fermentation tank at the end of the run. The term “Productivity” designates the quotient of the total L-threonine formed per fermentation run and the fermentation time per run.

TABLE 9
Composition of growth medium M1-531

| | |
|---|---|
| Component | Concentration (per kg) |

| | | |
|---|---|---|
| Corn steep liquor (50%) | 22.3 | g |
| Citric acid | 0.214 | g |
| (NH₄)₂SO₄ | 0.56 | g |
| KH₂PO₄ | 2.79 | g |
| MgSO₄ · 7H₂O | 2.23 | g |
| MnSO₄ · H₂O | 24 | mg |
| FeSO₄ · 7H₂O | 34 | mg |
| Kanamycin | 50 | mg |
| Structol | 0.3 | g |

TABLE 10
Results from Example 4

| | | | | total L- | |
|---|---|---|---|---|---|
| | | | OD | Threonine | Product- |
| | | L-Threonine | (660 | formed | ivity |
| Run | Time [h] | [g/l] | nm) | [g] | [g/h] |
| 1 | 65.0 | 101.3 | 46.4 | 159.5 | 2.45 |
| 2 | 59.0 | 102.6 | 49.0 | 147.9 | 2.51 |

### COMPARATIVE EXAMPLE C

Preparation of L-Threonine with the Aid of the Escherichia coli K-12 Strain B-3996 by Conventional Fermentation

The L-threonine-producing E. coli strain B-3996 is described in U.S. Pat. No. 5,175,107 and deposited at the Russian National Collection for Industrial Microorganisms (VKPM, Moscow, Russia).

The strain B-3996 has, inter alia, an enhanced, “feed back” resistant aspartate kinase I-homoserine dehydrogenase I, an attenuated threonine deaminase, an attenuated threonine dehydrogenase, a resistance to at least 5 g/l L-threonine and the ability to utilize sucrose as a source of carbon.

An individual colony of the strain B-3996 was transinoculated on to minimal medium with the following composition: 3.5 g/l Na₂HPO₄*2H₂O, 1.5 g/l KH₂PO₄, 1 g/l NH₄Cl, 0.1 g/l MgSO₄*7H₂O, 2 g/l sucrose, 20 g/l agar, 20 μg/ml streptomycin. The culture was incubated at 37° C. for approx. 5 days. 10 ml preculture medium with the following composition: 2 g/l yeast extract, 10 g/l (NH₄)₂SO₄, 1 g/l KH₂PO₄, 0.5 g/l MgSO₄*7H₂O, 15 g/l CaCO₃, 20 g/l sucrose, 20 μg/ml streptomycin were inoculated with an inoculating loop and incubated for 16 h at 37° C. and 180 rpm on an ESR incubator from Kuhner AG (Birsfelden, Switzerland).

A volume of 20 ml of this first preculture was inoculated into 1000 g of the nutrient medium A1-160. The culturing fermentation was carried out in 2 l stirred reactor fermenters from B. Braun (BBI, Germany, Melsungen, Biostat MD model). The nutrient medium A1-160 contained the constituents listed in Table 11. This second preculture was cultured for 14 h at a temperature of 37° C., a volume-specific gassing of 1.00 vvm, an oxygen partial pressure of 10% of the atmospheric saturation and a pH of pH 6.9 until all the sucrose had been consumed.

For inoculation of 1000 g of the growth medium M1-546, which was contained in 2 l stirred reactor fermenters from B. Braun (BBI, Germany, Melsungen, Biostat MD model), 100 g of the second preculture in nutrient medium A1-160 were added. The growth medium M1-546 contained the constituents listed in Table 12. The culture was cultured at a temperature of 37° C., an aeration of 1.0 l/min, a minimum stirring of 800 rpm and a pH of 6.9 and an oxygen partial pressure of 20% of the atmospheric saturation until all the sucrose initially introduced had been consumed after 7 h. The fermentation broth obtained in this way was then cultured for a further 29 h at a temperature of 37° C., an oxygen partial pressure of 20% of the atmospheric saturation, an aeration of 1.0 l/min and a pH of pH 6.9. During this time, a production medium comprising a sucrose solution with a concentration of 600 g/kg was fed in such that the sucrose concentration was always above 0.5 g/l.

The optical density (OD) was then determined with a digital photometer of the LP1W type from Dr. Bruno Lange GmbH (Berlin, Germany) at a measurement wavelength of 660 nm and the concentration of L-threonine formed was determined by ion exchange chromatography and post-column reaction with ninhydrin detection with an amino acid analyzer from Eppendorf-BioTronik (Hamburg, Germany),

63.8 g L-threonine were produced in the fermentation in 36 h. The productivity in this experiment was thus 1.77 g/h.

TABLE 11
Composition of nutrient medium M1-160

| | |
|---|---|
| Component | Concentration (per kg) |

| | | |
|---|---|---|
| Sucrose | 40 | g |
| Yeast extract | 2 | g |
| (NH₄)₂SO₄ | 5 | g |
| K₂HPO₄ | 2 | g |
| MgSO₄ · 7H₂O | 0.4 | g |
| FeSO₄ · 7H₂O | 20 | mg |
| MnSO₄ · H₂O | 20 | mg |
| Streptomycin | 100 | mg |
| Structol | 0.2 | g |

TABLE 12
Composition of growth medium M1-546

| | |
|---|---|
| Component | Concentration (per kg) |

| | | |
|---|---|---|
| Sucrose | 30 | g |
| Yeast extract | 2 | g |
| (NH₄)₂SO₄ | 5 | g |
| K₂HPO₄ | 2 | g |
| MgSO₄ · 7H₂O | 0.4 | g |
| MnSO₄ · H₂O | 20 | mg |
| FeSO₄ · 7H₂O | 20 | mg |
| NaCl | 0,6 | |
| Structol | 0.3 | g |

### EXAMPLE 5

Preparation of L-Threonine with the Aid of the Escherichia coli K-12 Strain B-3996 with 5 Subsequent Feed Processes and 10% Inoculation in Each Case

For inoculation of 1000 g of the growth medium M1-546, which was contained in 2 l stirred reactor fermenters from B. Braun (BBI, Germany, Melsungen, Biostat MD model), 100 g of the second preculture in nutrient medium A1-160 were added, as described in comparative example C. The growth medium M1-546 contained the constituents listed in Table 12. The culture was cultured at a temperature of 37° C., an aeration of 1.0 l/min, a minimum stirring of 800 rpm and a pH of 6.9 and an oxygen partial pressure of 20% of the atmospheric saturation until all the sucrose initially introduced had been consumed after 7 h. The fermentation broth obtained in this way was then cultured for a further 29 h at a temperature of 37° C., an oxygen partial pressure of 20% of the atmospheric saturation, an aeration of 1.0 l/min and a pH of pH 6.9. During this time, 411.8 g of a production medium comprising a sucrose solution with a concentration of 600 g/kg was fed in continuously.

The fermentation broth was then drained off to 10% of the total amount. The remaining 10% of the total amount was topped up with growth medium M1-546 to the starting weight of 1100 g and the fermentation was started again. The growth medium M1-546 contained the constituents listed in Table 13. The culture of this third run was cultured as described in Example 7 at a temperature of 37° C., an aeration of 1 l/min, a minimum stirring of 800 rpm and a pH of 6.9 and an oxygen partial pressure of 20% of the atmospheric saturation until all the sucrose initially introduced had been consumed after 8 h. The culture was then cultured for a further 28 h at a temperature of 37° C., an oxygen partial pressure of 20% of the atmospheric saturation and a pH of pH 6.9. During this time, a production medium comprising a sucrose solution with a concentration of 600 g/kg was fed in such that the sucrose concentration in the fermenter was always above 0.5 g/l. After a total of 36 h, the fermentation run was ended. The draining off of the fermentation broth to 10% and the topping up of the fermenter with M1-546 was repeated a total of five times.

At the end of each fermentation the OD and the concentration of L-threonine formed were determined as in Comparative Example A.

Table 13 shows the results of the particular runs. The term “Total L-threonine formed” relates to the L-threonine effectively formed or produced during the fermentation run. To calculate the total L-threonine formed, the amount of L-threonine introduced by the inoculum is subtracted from the amount of L-threonine present in the fermentation tank at the end of the run. The term “Productivity” designates the quotient of the total L-threonine formed per fermentation run and the fermentation time per run.

TABLE 13
Results from Example 5

| | | L-Threonine | | Total L- | |
|---|---|---|---|---|---|
| | | yield | OD | Threonine | Product- |
| | | [g/g | (660 | formed | ivity |
| Run | Time [h] | sucrose] | nm) | [g] | [g/h] |
| 1 | 36 | 22.7 | 39.1 | 63.8 | 1.77 |
| 2 | 36 | 25.9 | 26.2 | 45.2 | 1.26 |
| 3 | 36 | 19.9 | 39.0 | 47.5 | 1.32 |
| 4 | 36 | 27.5 | 40.0 | 79.3 | 2.20 |
| 5 | 36 | 21.3 | 40.0 | 63.2 | 1.76 |
| 6 | 36 | 26.3 | 41.7 | 82.4 | 2.29 |

### REFERENCE EXAMPLE D

Production of L-Threonine with the Aid of the Escherichia coli Strain KCCM 10132, by Means of Conventional Fermentation

The L-threonine-producing E. coli strain KCCM 10132 is described in WO 00/09660 and filed with the Korean Culture Center of Microorganisms (Korean Culture Center of Microorganisms, Department of Food Engineering & College of Engineering, Yonsei University Sodaemun-gu, Seoul, Korea).

The KCCM 10132 strain is resistant to, among other things, L-threonine and L-glutamic acid, as well as to α-methyl serine and diaminosuccinate, and is sensitive to fluoropyruvate. In addition, KCCM 10132 needs L-isoleucine and L-methionine for growth.

A single colony of the KCCM 10132 strain was inoculated in 10 ml of preculture medium with the following composition and incubated for 20 hours at 30° C. and 120 rpm on an ESR incubator manufactured by Kuhner AG (Birsfelden, Switzerland): 10 g/l yeast extract, 10 g/l peptone, 5 g/l NaCl, 3 g/l meat extract, 5 g/l glucose.

A volume of 9 ml of this first preculture was inoculated in 900 g of A1-171 culture medium. The culture fermentation was completed in 2 l agitating reactor fermenters manufactured by the B. Braun Company (BBI, Melsungen, Germany, Biostat MD model). The A1-171 culture medium contained the components listed in Table 14. This second preculture was cultured for 21.5 hours at a temperature of 30° C., volume-specific gassing of 0.9 vvm, oxygen partial pressure of 10% of air saturation, and a pH value of 7.0 until the glucose was completely consumed.

To inoculate 1000 g of the M1-626 growth medium, which was contained in 2 l agitating reactor fermenters manufactured by the B. Braun Company (BBI, Melsungen, Germany, Biostat MD model), 20 g of the second preculture in A1-171 culture medium were added. The M1-626 growth medium contained the components listed in Table 15. The culture was cultured for 27 hours at a temperature of 30° C., aeration of 0.8 l/min, minimum agitation of 800 rpm, a pH value of 7.0, and oxygen partial pressure of 20% of air saturation. The fermentation broth obtained in this manner was subsequently cultured for an additional 45 hours at a temperature of 30° C., oxygen partial pressure of 20% of air saturation, aeration of 1.5 l/min, and a pH value of 7.0. 207 g of a production medium composed of a glucose solution at a concentration of 660 g/kg were continuously added during this period.

Subsequently, the optical density (OD) was determined with a digital photometer, model LP1W manufactured by Dr. Bruno Lange GmbH (Berlin, Germany), at a measuring wavelength of 660 nm, and the concentration of L-threonine formed was determined with an amino acid analyzer manufactured by the Eppendorf-BioTronik company (Hamburg, Germany) using ion exchange chromatography and after-column reaction with ninhydrin detection.

After 72 hours, an L-threonine concentration of 49.4 g/l was determined in the final fermentation sample. Thus, productivity in this experiment comprised 0.69 g/l×hr.

TABLE 14
Composition of the A1-171 culture medium

| | |
|---|---|
| | Concentration |
| Component | (per kg) |

| | | |
|---|---|---|
| Glucose hydrate | 22 | g |
| Corn steep liquor (50%) | 30 | g |
| (NH₄)₂SO₄ | 0.5 | g |
| Urea | 6 | g |
| KH₂PO₄ | 1 | g |
| FeSO₄ · 7H₂O | 2 | mg |
| MnSO₄ · H₂O | 2 | mg |
| L-methionine | 200 | mg |
| L-isoleucine | 200 | mg |
| Struktol | 0.2 | g |

TABLE 15
Composition of the M1-626 growth medium

| | |
|---|---|
| | Concentration |
| Component | (per kg) |

| | | |
|---|---|---|
| Glucose hydrate | 110 | g |
| Corn steep liquor (50%) | 30 | g |
| (NH₄)₂SO₄ | 5 | g |
| KH₂PO₄ | 1 | g |
| MnSO₄ · H₂O | 2 | mg |
| FeSO₄ · 7H₂O | 2 | mg |
| L-methionine | 200 | mg |
| L-isoleucine | 200 | mg |
| Struktol | 0.3 | g |

### EXAMPLE 6

Production of L-Threonine with the Aid of the Escherichia coli Strain KCCM 10132, with a Downstream Additional Run Process and 10% Inoculation

To inoculate 1000 g of M1-626 growth medium, which was contained in 2 l agitating reactor fermenters manufactured by the B. Braun Company (BBI, Melsungen, Germany, Biostat MD model), 20 g of the second preculture in A1-171 were added as described in reference example D. The M1-626 growth medium contained the components listed in Table 15. The culture was cultured for 27 hours at a temperature of 30° C., aeration of 0.8 l/min, minimum agitation of 800 rpm, a pH value of 7.0, and oxygen partial pressure of 20% of air saturation. The fermentation broth obtained in this manner was subsequently cultured for an additional 45 hours at a temperature of 30° C., oxygen partial pressure of 20% of air saturation, aeration of 1.5 l/min, and a pH value of 7.0. 207 g of a production medium composed of a glucose solution at a concentration of 660 g/kg were continuously added during this period.

Subsequently, the fermentation broth was drained down to 10% of its total volume. M1-626 growth medium was added to the remaining 10% to arrive at the initial mass of 1020 g, and fermentation was restarted. The M1-626 growth medium contained the components listed in Table 15. This second run was cultured for 27 hours at a temperature of 30° C., aeration of 0.8 l/min, minimum agitation of 800 rpm, a pH value of 7.0, and oxygen partial pressure of 20% of air saturation. The culture was then cultured for an additional 41 hours at a temperature of 30° C., oxygen partial pressure of 20% of air saturation, aeration of 1.5 l/min, and a pH value of 7.0. 151 g of a production medium composed of a glucose solution at a concentration of 660 g/kg were continuously added during this period.

At the end of fermentation, the optical density (OD) was determined with a digital photometer, model LP1W manufactured by Dr. Bruno Lange GmbH (Berlin, Germany), at a measuring wavelength of 660 nm, and the concentration of L-threonine formed was determined with an amino acid analyzer manufactured by Eppendorf-BioTronik (Hamburg, Germany) using ion exchange chromatography and after-column reaction with ninhydrin detection.

The results of the respective runs are shown in Table 3. The term “total L-threonine formed” refers to the L-threonine actually formed or produced during the fermentation run. To calculate the total L-threonine formed, the amount of L-threonine introduced by the inoculum is subtracted from the amount of L-threonine present in the fermentation tank at the end of the run. The term “productivity” designates the quotient of the total L-threonine formed per fermentation run and the fermentation time per run.

TABLE 16

| | | L-threonine | | |
|---|---|---|---|---|
| | | yield | Total L-threonine | |
| | Time | [g/g | formed | Productivity |
| Run | [h] | sucrose] | [g] | [g/h] |
| 1 | 72 | 26.5 | 49.4 | 0.69 |
| 2 | 68 | 25.9 | 48.5 | 0.71 |

### REFERENCE EXAMPLE E

Production of L-Threonine with the Aid of the Escherichia coli Strain H-4581, by Means of Conventional Fermentation

The L-threonine-producing E. coli strain H-4581 is described in EP 301 572 and filed with the Fermentation Research Institute, Agency of Industrial Science and Technology (Japan) on Jul. 16, 1987, under number FERM BP-1411.

The H-4581 Escherichia coli strain is resistant to, among other things, rifampicine, lysine, methionine, aspartic acid, and homoserine.

A single colony of the H-4581 strain was inoculated in 10 ml of preculture medium with the following composition and incubated for 24 hours at 30° C. and 180 rpm on an ESR incubator manufactured by Kuhner AG (Birsfelden, Switzerland): 10 g/l tryptone, 5 g/l yeast extract, 5 g/l NaCl, 1 g/l glucose, and 0.5 g/l diaminopimelic acid.

A volume of 0.5 ml of this first preculture was inoculated in 700 ml of A1-172 culture medium. The culture fermentation was completed in 2 l agitating reactor fermenters manufactured by the B. Braun Company (BBI, Melsungen, Germany, Biostat MD model). The A1-172 culture medium contained the components listed in Table 4. This second preculture was cultured for 15 hours at a temperature of 30° C., volume-specific gassing of 1.0 vvm, oxygen partial pressure of at least 10% of air saturation, and a pH value of 7.0 until the glucose was completely consumed.

To inoculate 1000 g of the M1-627 growth medium, which was contained in 2 l agitating reactor fermenters manufactured by the B. Braun Company (BBI, Melsungen, Germany, Biostat MD model), 100 g of the second preculture in A1-172 culture medium were added. The M1-627 growth medium contained the components listed in Table 17. The culture was cultured for 5.75 hours at a temperature of 30° C., aeration of 1.0 l/min, minimum agitation of 800 rpm, a pH value of 6.5, and oxygen partial pressure of 20% of air saturation. The fermentation broth obtained in this manner was subsequently cultured for an additional 69.75 hours at a temperature of 30° C., oxygen partial pressure of 20% of air saturation, aeration of 1.5 l/min, and a pH value of 6.5. 267 g of a production medium composed of a glucose solution at a concentration of 660 g/kg were continuously added during this period.

Subsequently, the optical density (OD) was determined with a digital photometer, model LP1W manufactured by Dr. Bruno Lange GmbH (Berlin, Germany), at a measuring wavelength of 660 nm, and the concentration of L-threonine formed was determined with an amino acid analyzer manufactured by Eppendorf-BioTronik (Hamburg, Germany) using ion exchange chromatography and after-column reaction with ninhydrin detection.

After 75.5 hours, an L-threonine concentration of 6.95 g/l was determined in the final fermentation sample. Thus, productivity in this experiment comprised 92.1 mg/l×hr.

TABLE 17
Composition of the A1-172 culture medium

| | |
|---|---|
| | Concentration |
| Component | (per kg) |

| | |
|---|---|
| Glucose hydrate | 22 g |
| Casein peptone | 10 g |
| NaCl | 2.5 g |
| Yeast extract | 10 g |
| Diaminopimelic acid | 0.1 g |
| Struktol | 0.1 g |

TABLE 18
Composition of the M1-627 growth medium

| | |
|---|---|
| | Concentration |
| Component | (per kg) |

| | | |
|---|---|---|
| Glucose hydrate | 33 | g |
| Corn steep liquor (50%) | 12 | g |
| (NH₄)₂SO₄ | 12 | g |
| KH₂PO₄ | 3 | g |
| MgSO₄ · 7H₂O | 0.2 | mg |
| Diaminopimelic acid | 0.6 | g |
| L-methionine | 50 | mg |
| Struktol | 0.1 | g |

### EXAMPLE 7

Production of L-Threonine with the Aid of the Escherichia coli Strain H-4581, with a Downstream Additional Run Process and 10% Inoculation

To inoculate 1000 g of M1-627 growth medium, which was contained in 2 l agitating reactor fermenters manufactured by the B. Braun Company (BBI, Melsungen, Germany, Biostat MD model), 100 g of the second preculture in A1-172 were added as described in reference example E. The M1-627 growth medium contained the components listed in Table 18. The culture was cultured for 5.75 hours at a temperature of 30° C., aeration of 1.0 l/min, minimum agitation of 800 rpm, a pH value of 6.5, and oxygen partial pressure of 20% of air saturation. The fermentation broth obtained in this manner was subsequently cultured for an additional 69.75 hours at a temperature of 30° C., oxygen partial pressure of 20% of air saturation, aeration of 1.5 l/min, and a pH value of 6.5. 267 g of a production medium composed of a glucose solution at a concentration of 660 g/kg were continuously added during this period.

Subsequently, the fermentation broth was drained down to 10% of its total volume. M1-627 culture medium was added to the remaining 10% to arrive at the initial mass of 1100 g, and fermentation was restarted. The M1-627 growth medium contained the components listed in Table 18. This second run was cultured for 4.25 hours at a temperature of 30° C., aeration of 1.0 l/min, minimum agitation of 800 rpm, a pH value of 6.5, and oxygen partial pressure of 20% of air saturation. The culture was then cultured for an additional 71.5 hours at a temperature of 30° C., oxygen partial pressure of 20% of air saturation, aeration of 1.5 l/min, and a pH value of 7.0. 225 g of a production medium composed of a glucose solution at a concentration of 660 g/kg were continuously added during this period.

At the end of fermentation, the optical density (OD) was determined with a digital photometer, model LP1W manufactured by Dr. Bruno Lange GmbH (Berlin, Germany), at a measuring wavelength of 660 nm, and the concentration of L-threonine formed was determined with an amino acid analyzer manufactured by Eppendorf-BioTronik (Hamburg, Germany) using ion exchange chromatography and after-column reaction with ninhydrin detection.

The results of the respective runs are shown in Table 19. The term “total L-threonine formed” refers to the L-threonine actually formed or produced during the fermentation run. To calculate the total L-threonine formed, the amount of L-threonine introduced by the inoculum is subtracted from the amount of L-threonine present in the fermentation tank at the end of the run. The term “productivity” designates the quotient of the total L-threonine formed per fermentation run and the fermentation time per run.

TABLE 19

| | | Total L-threonine | |
|---|---|---|---|
| | | formed | Productivity |
| Run | Time [h] | [g] | [mg/h] |
| 1 | 75.5 | 7.92 | 104.9 |
| 2 | 72.75 | 10.02 | 137.7 |

What is claimed is:

1. Process for the fermentative preparation of L-threonine, wherein
a) an L-threonine-producing microorganism of the Enterobacteriaceae family is cultured by the feed process (fed batch), subsequently
b) a portion of the fermentation broth is separated off, 1 to 90 vol. %, in particular 1 to 50 vol. %, preferably 1 to 25 vol. % of the total volume of the fermentation broth remaining in the fermentation tank, subsequently
c) the remaining fermentation broth is topped up with growth medium and, preferably after a growth phase, a further fermentation is carried out in accordance with a),
d) steps b) and c) are optionally carried out several times, and
e) the L-threonine is isolated from the fermentation broths collected.

2. Process according to claim 1, wherein steps b) and c) are carried out two to six times and the L-threonine is isolated from the fermentation broths collected.

3. Process according to claims 1 and 2, wherein microorganisms of the species Escherichia coli are employed.

4. L-Threonine-producing and -secreting microorganisms of the Enterobacteriaceae family which have an enhanced resistant aspartate kinase I-homoserine dehydrogenase I, an attenuated threonine deaminase, a resistance to at least 5 g/l threonine and the ability to utilize sucrose as a source of carbon, and the parB gene region.

5. Transformants according to claim 4, deposited under number DSM 12790 at the DSMZ [German Collection of Microorganisms and Cell Cultures], Braunschweig.

6. Process according to claims 1 and 2, wherein L-threonine-producing and -secreting microorganisms of the Enterobacteriaceae family which have one or more of the features chosen from the group consisting of: an enhanced, feed back resistant aspartate kinase I-homoserine dehydrogenase I, an attenuated threonine deaminase, an attenuated threonine dehydrogenase, a resistance to at least 5 g/l threonine, a resistance to borrelidin, a resistance to α-methylserine, a resistance to diaminosuccinic acid, a sensitivity to fluoropyruvate, a resistance to L-glutamic acid, a need for L-methionine, and the ability to utilize sucrose as a source of carbon are used.

7. Process according to claim 6, wherein L-threonine-producing and -secreting microorganisms of the Enterobacteriaceae family which have an enhanced resistant aspartate kinase I-homoserine dehydrogenase I, an attenuated threonine deaminase, a resistance to at least 5 g/l threonine and the ability to utilize sucrose as a source of carbon are used.

8. Process according to claims 1 and 2, wherein L-threonine-producing and -secreting microorganisms of the Enterobacteriaceae family which have one or more of the features chosen from the group consisting of: an enhanced, feed back resistant aspartate kinase I-homoserine dehydrogenase I, an attenuated threonine deaminase, an attenuated threonine dehydrogenase, a resistance to at least 5 g/l threonine and the ability to utilize sucrose as a source of carbon are used.

9. Process according to claims 1 and 2, wherein L-threonine-producing and -secreting microorganisms of the Enterobacteriaceae family which have one or more of the features chosen from the group consisting of: an enhanced, feed back resistant aspartate kinase I-homoserine dehydrogenase I, an attenuated threonine deaminase, a resistance to borrelidin and the ability to utilize sucrose as a source of carbon are used.

10. Process according to claims 1 and 2, wherein L-threonine-producing and -secreting microorganisms of the Enterobacteriaceae family which have one or more of the features chosen from the group consisting of: a resistance to α-methylserine, a resistance to diaminosuccinic acid, a sensitivity to fluoropyruvate, a resistance to L-glutamic acid and a resistance to at least 7% L-threonine, a need for L-methionine and a need for L-isoleucine are used.

11. Process according to claims 1 and 2, wherein L-threonine-producing and -secreting microorganisms of the Enterobacteriaceae family which have one or more of the features or characteristics of the strains chosen from the group consisting of: DSM12790, B-3996, kat 13, KCCM-1032 and KCCM-1033 are used.

12. Process according to claims 1 and 2, wherein one or more strains chosen from the group consisting of DSM12790, B-3996, kat 13, KCCM-1032 and KCCM-1033 are used.
